(19) United States
(12) Patent Application Publication
(10) Pub. No.: US 2020/0069685 A1
(43) Pub. Date: Mar. 5, 2020

(54) Dosing schedule for a combination of Ceritinib and an anti-PD-1 antibody molecule

(71) Applicants: NOVARTIS AG, Basel (CH); BRISTOL-MYERS SQUIBB COMPANY, New York, NY (US)

(72) Inventor: Jeffrey SCOTT, East Hanover, NJ (US)

(21) Appl. No.: 16/489,088

(22) PCT Filed: Feb. 26, 2018
(86) PCT No.: PCT/IB2018/051184

§ 371 Date: Aug. 27, 2019

Related U.S. Application Data
(60) Provisional application No. 62/463,871, filed on Feb. 27, 2017.

(51) Int. Cl. A61K 31/506; A61K 39/395; A61P 35/00

(52) U.S. Cl. CPC A61K 31/506; A61K 39/3955; A61K 2039/505; A61P 35/00; A61K 39/39558

(57) ABSTRACT

The present disclosure relates to the field of pharmacy, particularly to ceritinib and a PD-1 inhibitor for use in the treatment of cancer. Specifically, the disclosure relates to a pharmaceutical combination comprising ceritinib, or a pharmaceutically acceptable salt thereof, and a PD-1 inhibitor, or a pharmaceutically acceptable salt thereof, for use in the treatment of cancer, to a method for the treatment of cancer that involves administering the combination and to the use of the combination for the manufacture of a medicament for the treatment of cancer.

### FIELD OF THE DISCLOSURE

The present disclosure relates to the field of pharmacy, particularly to ceritinib and an anti-PD-1 antibody molecule for use in the treatment of cancer. Specifically, the disclosure relates to a pharmaceutical combination comprising ceritinib, or a pharmaceutically acceptable salt thereof, and an anti-PD-1 antibody molecule, or a pharmaceutically acceptable salt thereof, for use in the treatment of cancer; to a method for the treatment of cancer that involves administering the combination; and to the use of the combination for the manufacture of a medicament for the treatment of cancer.

### BACKGROUND OF THE DISCLOSURE

Anaplastic lymphoma kinase (ALK) is a receptor tyrosine kinase (RTK) belonging to the insulin receptor superfamily. Genetic alterations of ALK have been implicated in oncogenesis in hematopoietic and non-hematopoietic tumors. The gene has been found to be rearranged, mutated, or amplified in a series of tumors, including non-small cell lung cancer (NSCLC). The identification of ALK as an oncogene has led to the development of ALK tyrosine kinase inhibitors, such as crizotinib, or ceritinib, and their deployment in a treatment of cancers harboring ALK mutations, rearrangements or amplifications, in particular ALK-positive NSCLC.

The Programmed Death 1 (PD-1) protein is an inhibitory member of the extended CD28/CTLA4 family of T-cell regulators (Okazaki et al. Curr. Opin. Immunol. 2002, 14, 391779; Bennett et al. J. Immunol. 2003, 170, 711). Ligands of the CD28 receptor include a group of related B7 molecules, also known as the “B7 Superfamily” (Coyle et al. Nature Immunol. 2001, 2(3), 203; Sharpe et al. Nature Rev. Immunol. 2002, 2, 116; Collins et al. Genome Biol. 2005, 6, 223.1; Korman et al. Adv. Immunol. 2007, 90, 297). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins et al. Genome Biol. 2005, 6, 223.1). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

PD-L1 is abundant in a variety of human cancers (Dong et al. Nat. Med. 2002, 8, 787). PD-1 is known as an immune-inhibitory protein that negatively regulates TCR signals (Ishida et al. EMBO J. 1992, 11, 3887; Blank et al. Immunol. Immunother. 2006, 56(5), 739). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. J. Mol. Med. 2003, 81, 281; Blank et al. Cancer Immunol. Immunother. 2005, 54, 307; Konishi et al. Clin. Cancer Res. 2004, 10, 5094). Immune suppression can be reversed by inhibiting the local interaction of PD-1 with PD-L1 or PD-L2; the effect is additive when the interaction of PD-1 with PD-L2 is blocked as well (Iwai et al. Proc. Nat. Acad. Sci. USA 2002, 99:12293-7; Brown et al. J. Immunol. 2003, 170, 1257).

### SUMMARY OF THE DISCLOSURE

Given the importance of immune checkpoint pathways in regulating an immune response in cancer therapy, the need exists to develop novel combination therapies that activate the immune system or overcome the resistance to targeted therapies.

The invention addresses this need by providing a pharmaceutical combination as defined herein.

The first aspect of the present disclosure relates to a pharmaceutical combination comprising (i) ceritinib or a pharmaceutically acceptable salt thereof, and (ii) anti-PD-1 antibody molecule or a pharmaceutically acceptable salt thereof, for use in the treatment of cancer, wherein (i) ceritinib is administered as monotherapy for 2 cycles before a combination therapy with the (ii) anti-PD-1 antibody molecule is initiated.

Another aspect of the present disclosure provides a method for the treatment of a patient, wherein a therapeutically effective amount of ceritinib (i) and (ii) anti-PD-1 antibody molecule is administered to the patient as defined in any one of claims 1 to 16 herein.

Yet another aspect relates to the use of the combination as defined in any one of the claims 1 to 16 for the manufacture of a medicament.

The “monotherapy” relates only to the administration of ceritinib prior to administration of an ii) anti-PD-1 antibody molecule or a pharmaceutically acceptable salt thereof and is not meant to limit the use of ceritinib with regard to any concomitant use of an active ingredient other that an anti-PD-1 antibody molecule, e.g. any adjuvant therapy or the like.
Furthermore, the present disclosure provides the following aspects, advantageous features and specific embodiments, respectively alone or in combination, as listed in the following items:
1. Ceritinib for use in the treatment of cancer in combination with an anti-PD-1 antibody molecule, wherein ceritinib is administered for 2 cycles before initiation of combination therapy with the anti-PD-1 antibody molecule.
2. An anti-PD-1 antibody molecule for use in the treatment of cancer in combination with ceritinib, wherein ceritinib is administered for 2 cycles before initiation of combination therapy with the anti-PD-1 antibody molecule.
3. Ceritinib for use in the treatment of cancer according to item 1 or an anti-PD-1 antibody molecule for use in the treatment of cancer according to item 2, wherein the combination therapy with the anti-PD-1 antibody molecule is initiated only in a patient without rash of any grade.
4. Ceritinib for use in the treatment of cancer according to items 1 or 3, or an anti-PD-1 antibody molecule for use in the treatment of cancer according to items 2 or 3, wherein the patient has Aspartate aminotransaminase (AST)≤3×ULN, Alanine aminotransaminase (ALT)≤3×ULN and Alkaline phosphatase (ALP)≤5.0×ULN.
5. Ceritinib for use in the treatment of cancer according to items 1, 3 or 4, or an anti-PD-1 antibody molecule for use in the treatment of cancer according to any one of items 2 to 4, wherein (i) ceritinib is administered for 3 cycles before initiation of combination therapy with the anti-PD-1 antibody molecule.
6. Ceritinib for use in the treatment of cancer according to any one of items 1, or 3 to 5, or an anti-PD-1 antibody molecule for use in the treatment of cancer according to any one of items 2 to 5, wherein ceritinib is administered for at least 7 consecutive days prior to the first dose of the (ii) anti-PD-1 antibody molecule.
7. Ceritinib for use in the treatment of cancer according to any one of items 1, or 3 to 6, or an anti-PD-1 antibody molecule for use in the treatment of cancer according to any one of items 2 to 6, wherein (i) ceritinib is administered at a starting dose of 450 mg ceritinib daily together with a low-fat meal.
8. Ceritinib for use in the treatment of cancer according to item 7, or an anti-PD-1 antibody molecule for use in the treatment of cancer according to item 7, wherein ceritinib starting dose is reduced to 375 mg, 300 mg or 150 mg daily.
9. Ceritinib for use in the treatment of cancer according to items 7 or 8, or an anti-PD-1 antibody molecule for use in the treatment of cancer according to items 7 or 8, wherein the ceritinib starting dose is reduced to 375 mg.
10. Ceritinib for use in the treatment of cancer according to any one of items 1, or 3 to 9, or an anti-PD-1 antibody molecule for use in the treatment of cancer according to any one of items 2 to 9, wherein the cancer is ALK-positive cancer.
11. Ceritinib for use in the treatment of cancer according to item 10, or an anti-PD-1 antibody molecule for use in the treatment of cancer according to item 10, wherein the cancer is NSCLC.
12. Ceritinib for use in the treatment of cancer according to any one of items 1, or 3 to 11, or an anti-PD-1 antibody molecule for use in the treatment of cancer according to any one of items 2 to 11, wherein the (ii) anti-PD-1 antibody molecule is selected from nivolumab, pembrolizumab, pidilizumab, PDR-001, or a pharmaceutical salt thereof.
13. Ceritinib for use in the treatment of cancer according to any one of items 1, or 3 to 12, or an anti-PD-1 antibody molecule for use in the treatment of cancer according to any one of items 2 to 12, wherein the (ii) anti-PD-1 antibody molecule is administered every 2 weeks, every 3 weeks or every 4 weeks.
14. Ceritinib for use in the treatment of cancer according to item 13, or an anti-PD-1 antibody molecule for use in the treatment of cancer according to item 13, wherein the (ii) anti-PD-1 antibody molecule is nivolumab and 3 mg/kg nivolumab is administered every 2 weeks.
15. Ceritinib for use in the treatment of cancer according to any one of items 1, or 3 to 13, or an anti-PD-1 antibody molecule for use in the treatment of cancer according to any one of items 2 to 13, wherein the (ii) anti-PD-1 antibody molecule is PDR-001, or a pharmaceutical salt thereof.
16. Ceritinib for use in the treatment of cancer according to item 15, or an anti-PD-1 antibody molecule for use in the treatment of cancer according to item 15, wherein PDR-001 is administered intravenously in a single dose of 300 to 400 mg/day.
17. Ceritinib for use in the treatment of cancer according to any one of items 1, or 3 to 16, or an anti-PD-1 antibody molecule for use in the treatment of cancer according to any one of items 2 to 16, wherein the patient is Asian.

### DETAILED DESCRIPTION OF THE DISCLOSURE

The present disclosure relates to a specific dosing regimen for a combination therapy involving ceritinib. Ceritinib is an ALK inhibitor with the chemical name 5-chloro-N2-(2-isopropoxy-5-methyl-4-(piperidin-4-yl)phenyl)-N4-[2-(propane-2-sulfonyl)-phenyl]-pyrimidine-2,4-diamine, or a pharmaceutically acceptable salt thereof. The compound 5-chloro-N2-(2-isopropoxy-5-methyl-4-(piperidin-4-yl)phenyl)-N4-[2-(propane-2-sulfonyl)-phenyl]-pyrimidine-2,4-diamine, i.e. ceritinib, is a compound of formula I, and is described in Example 7 (Compound 66) of WO2008/073687. It is commercially available as Zykadia®.

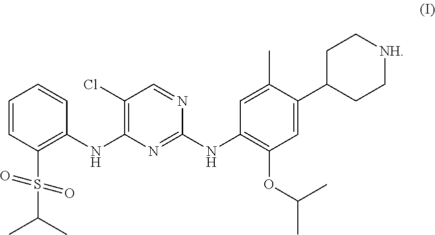 [SMILES: Cc1cc(Nc2ncc(Cl)c(Nc3ccccc3S(=O)(=O)C(C)C)n2)c(OC(C)C)cc1C1CCNCC1]

When using ceritinib alone, side effects such as nausea, diarrhea, vomiting, fatigue, ALT/AST elevation, constipation, abdominal pain were observed. Rash was observed rarely and particularly was easy-to-manage and self-limiting in population receiving ceritinib doses lower than 500 mg fasted. Rash did not require any dose reduction as opposed to other side effects. The doses were reduced from 750 mg in the steps of 150 mg down to the lowest 300 mg. If the patient required doses lower than 300 mg, ceritinib was discontinued. Approximately 54% of patients initiating treatment at the recommended dose of 750 mg fasted required at least one dose adjustment due to adverse reaction, with a median time to first dose reduction of approximately 7 weeks.
However, based on the preliminary data from 36 patients, who were enrolled to 4 cohorts to the concomitant combination therapy of ceritinib and nivolumab, the rates of severe rash (even grade 3) were unexpectedly higher. The cohorts were as follows: cohort 1 (n=8), ceritinib 450 mg daily with a low fat meal in combination with nivolumab 3 mg/kg Q2W; cohort 2 (n=6), ceritinib 450 mg daily with a low fat meal in combination with nivolumab 3 mg/kg Q2W; cohort 3 (n=11), ceritinib 300 mg daily with a low fat meal in combination with nivolumab 3 mg/kg Q2W; cohort 4 (n=11), ceritinib 300 mg daily with a low fat meal in combination with nivolumab 3 mg/kg Q2W.
Both dose levels, i.e. 450 mg and 300 mg of ceritinib, administered with the low fat meal led to severe rash. The 450 mg dose level had higher rates of some adverse events, especially grade-3 rash, than expected based on data from either single agent. The proportion of patients with grade-3 rash was higher at the 450 mg dose level, 4/14 patients (29%), than at the 300 mg dose level, 3/22 patients (14%).
With regard to the efficacy of the combination therapy, of the 14 patients treated at the 450 mg dose level, 9 (64%) achieved a confirmed partial response. Of the 22 patients treated at the 300 mg dose level, 4 (18%) achieved a confirmed complete (1 patient) or partial (3 patients) response; however, limited follow up, especially for cohort 4, confounded the assessment of confirmed response. The disease control rate (DCR) was similar for the 450 mg and 300 mg dose levels, 79% and 82% respectively. Thus, preliminary evidence of efficacy has been demonstrated at both tested dose levels. However, the 300 mg dose level appeared to have a lower number of objective responses, although this observation was based on a small number of patients with limited follow-up. In addition, preliminary ceritinib steady state AUC0-24 and Cmax suggested that at 300 mg administered with food, exposure was approximately 60-65% that of 450 mg administered with food.
Therefore, based on the safety, efficacy, and PK data a new beneficial dosing regimen is provided that is expected to improve tolerability while maintaining exposure similar to that observed at the 450 mg dose level. We provide herein a dosing regimen for the combination therapy of ceritinib and an anti-PD-1 antibody molecule, where ceritinib is administered for 2 cycles before initiation of combination therapy with nivolumab. The two cycles of ceritinib monotherapy administration should allow for safety observation and dose reduction, if required due to toxicity, to ensure that the dose of ceritinib is tolerable prior to initiation of combination therapy with an anti-PD-1 antibody molecule, such as nivolumab. Without wishing to be bound to any theory, the 2 cycles of ceritinib monotherapy should allow a patient to adapt to ceritinib and any local inflammation that ceritinib may have caused to fade out and thus led to reduced incidence or severity of rash. Additional time without an anti PD-1 antibody should allow immune system to adapt and cumulatively result in the better tolerability of the combination of ceritinib and anti PD-1 antibody. Rash is expected to be less pronounced and easier to manage.
According to the present disclosure ceritinib is administered for 2 cycles before initiation of combination therapy with nivolumab. Patients can take oral ceritinib at approximately the same time, preferably in the morning each day at the dose between 150 mg and 450 mg within 30 minutes after consuming a low-fat meal. The best practice would be for patients to refrain from eating again for at least 1 hour after dosing. Based on the available data, general patient population can benefit from the new dosing regimen. Asians, who are most affected in terms of the prevalence and severity of the rash, can benefit the most.
A “low-fat meal” is defined herein as one containing approximately 1.5 to 15 grams of fat and approximately 100 to 500 total calories. A low-fat meal (approximately 330 calories and 9 grams of fat) or very low-fat light snack (containing approximately 100-300 calories and 1.5 grams of fat) led to a clinically meaningful ceritinib exposure increase. A food effect study was conducted in healthy subjects study. Compared to the fasted state, a low-fat meal (approximately 330 calories and 9 grams of fat) increased Cmax and AUCinf of a single oral dose of ceritinib (500 mg) in healthy subjects by 43% and 58%, respectively, whereas a high-fat meal (approximately 1000 calories and 58 grams of fat) increased Cmax and AUCinf by 41% and 73%, respectively. To further clarify if low fat content has an impact on the extent of ceritinib absorption, a food effect assessment with a very low-fat light snack (containing approximately 100-300 calories and 1.5 grams of fat) was also explored in a relative bioavailability study conducted in healthy subjects study. PK data from the light snack cohort showed that when a single 750 mg oral dose of ceritinib was administered with a light snack, the Cmax and AUCinf increased by 45% and 54% respectively, compared to the fasted condition. This magnitude of increase is similar to that caused by a low-fat meal, suggesting that even a very low-fat meal could lead to a clinically meaningful ceritinib exposure increase.
In the present disclosure the term “pharmaceutical combination” refers to a non-fixed combination. The term “non-fixed combination” means that the active ingredients, e.g. compound of formula (i), namely ceritinib, or a pharmaceutically acceptable salt thereof and an anti-PD-1 antibody molecule, or a pharmaceutically acceptable salt form, are both administered to a patient as separate entities either simultaneously or sequentially with no specific time limits, wherein such administration provides therapeutically effective levels of the two compounds in the body of the patient. In special embodiment, some limitations are imposed as described herein.
The terms “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. In will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.
The term “cycle” refers to a specific period of time expressed in days that is repeated on a regular schedule and is 28 days long.
The term “grade” herein refers to the severity of an adverse event, such as rash. The grades herein are termed according to the CTCAE v 4.03 (Common terminology criteria for adverse events v 4.03).
The term “patient” refers to a human being that would benefit biologically, medically or in quality of life from the treatment.
The term “dose” refers to a specified amount of a drug administered at one time. As used herein, the dose is the amount of the drug that elicits a therapeutic effect. Unless specified otherwise, the dose relates to the amount of the drug in its free form. In the event the drug is in the form of a pharmaceutically acceptable salt, the amount of the drug is increased proportionately compared to the amount of the drug in its free form. The dose would for example be declared on a product package or in a product information leaflet.
The term “Asian” refers herein to a person having origins in any of the original peoples of the Far East, Southeast Asia, or the Indian subcontinent, including, for example, Cambodia, China, India, Japan, Korea, Malaysia, Pakistan, the Philippine Islands, Thailand, and Vietnam. The definition is commensurate to the Collection of Race and Ethnicity Data in Clinical Trials, the Guidance for Industry and Food and Drug Administration Staff issued on Oct. 26, 2016.
The anti-PD-1 antibody molecule (ii), or a pharmaceutically acceptable salts thereof, that can be used in combination with ceritinib, can be selected from nivolumab (Opdivo), pembrolizumab (Keytruda), pidilizumab, MDX-1106, Merck 3475, CT-011, AMP-224, YW243.55.S70, MPDL3280A, MEDI-4736, MSB-0010718C, MDX-1105 (also known as BMS-936559, an anti-PD-LI antibody described in WO2007/005874) or PDR-001, or a pharmaceutical salt thereof. Antibody YW243.55.S70 (heavy and light chain variable region sequences shown in SEQ ID Nos. 20 and 21, respectively) is an anti-PD-LI described in WO 2010/077634, the anti-PD-1 antibody is Nivolumab. Alternative names for Nivolumab include MDX-1106, MDX-1106-04, ONO-4538, or BMS-936558.
The anti-PD-1 antibody Nivolumab has a CAS Registry Number: 946414-94-4). Nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449, EP2161336 and WO2006/121168. The heavy and light chain amino acid sequences of Nivolumab are as follows:

Heavy chain
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light chain
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335. The heavy and light chain amino acid sequences of Pembrolizumab are as follows:

| Heavy chain | |
|---|---|
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTIVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| RVVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Ligh chain | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218′ |

Pidilizumab (CT-011; Cure Tech) is a humanized IgG1k monoclonal antibody that was previously reported to bind to PD-1, but which is believed to bind to a different target. Pidilizumab and other humanized anti-PD-1 monoclonal antibodies are disclosed in WO2009/101611.
MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.S70 (heavy and light chain variable regions are shown in SEQ ID NOs 20 and 21 in WO2010/077634) and MDX-1105 (also referred to as BMS-936559, and, e.g., anti-PD-L1 binding agents disclosed in WO2007/005874).

AMP-224 (B7-DCIg; Amplimmune; e.g., disclosed in WO2010/027827 and WO2011/066342), is a PD-L2 Fc fusion soluble receptor that blocks the interaction between PD1 and B7-H1.

The anti-PD-1 antibody molecule can comprise at least one antigen-binding region, e.g., a variable region or an antigen-binding fragment thereof, from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD1 antibody molecule is preferably selected from nivolumab (Opdivo), pembrolizumab (Keytruda), pidilizumab, PDR-001, or a pharmaceutical salt thereof.

The anti-PD-1 antibody molecule designated as PDR-001 was described in PCT/CN2016/099494. More particularly the PDR-001 inhibitor, or a pharmaceutically acceptable salt thereof, comprises a heavy chain variable region (VH) comprising a HCDR1, a HCDR2 and a HCDR3 amino acid sequence of BAP049-Clone-E and a light chain variable region (VL) comprising a LCDR1, a LCDR2 and a LCDR3 amino acid sequence of BAP049-Clone-E as described in Table 1.

The anti-PD-1 antibody molecule, or a pharmaceutically acceptable salt thereof, of the present disclosure comprises, for example, at least one, two, three or four variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure comprises, for example, at least one or two heavy chain variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure comprises, for example, at least one or two light chain variable regions from an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, of the present disclosure includes, for example, a heavy chain constant region for an IgG4, e.g., a human IgG4. The human IgG4 includes a substitution at position 228 according to EU numbering (e.g., a Ser to Pro substitution). The anti-PD-1 antibody molecule includes a heavy chain constant region for an IgG1, e.g., a human IgG1. The human IgG1 includes a substitution at position 297 according to EU numbering (e.g., an Asn to Ala substitution). The human IgG1 may also include a substitution at position 265 according to EU numbering, a substitution at position 329 according to EU numbering, or both (e.g., an Asp to Ala substitution at position 265 and/or a Pro to Ala substitution at position 329). The human IgG1 also includes a substitution at position 234 according to EU numbering, a substitution at position 235 according to EU numbering, or both (e.g., a Leu to Ala substitution at position 234 and/or a Leu to Ala substitution at position 235). The heavy chain constant region comprises an amino sequence set forth in Table 3, or a sequence substantially identical (e.g., at least 80%, 85%, 90%, 92%, 95%, 97%, 98%, 99% or higher identical) thereto.

The anti-PD-1 antibody molecule, or a pharmaceutically acceptable salt thereof, according to the present disclosure includes, for example, a kappa light chain constant region, e.g., a human kappa light chain constant region. The light chain constant region comprises an amino sequence set forth in Table 3, or a sequence substantially identical (e.g., at least 80%, 85%, 90%, 92%, 95%, 97%, 98%, 99% or higher identical) thereto.

The anti-PD-1 antibody molecule, or a pharmaceutically acceptable salt thereof, according to the present disclosure also includes, for example, a heavy chain constant region for an IgG4, e.g., a human IgG4, and a kappa light chain constant region, e.g., a human kappa light chain constant region, e.g., a heavy and light chain constant region comprising an amino sequence set forth in Table 3, or a sequence substantially identical (e.g., at least 80%, 85%, 90%, 92%, 95%, 97%, 98%, 99% or higher identical) thereto. The human IgG4 includes a substitution at position 228 according to EU numbering (e.g., a Ser to Pro substitution). The anti-PD-1 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 3, or a sequence substantially identical (e.g., at least 80%, 85%, 90%, 92%, 95%, 97%, 98%, 99% or higher identical) thereto. The human IgG1 may also include a substitution at position 297 according to EU numbering (e.g., an Asn to Ala substitution). The human IgG1 includes a substitution at position 265 according to EU numbering, a substitution at position 329 according to EU numbering, or both (e.g., an Asp to Ala substitution at position 265 and/or a Pro to Ala substitution at position 329). The human IgG1 includes a substitution at position 234 according to EU numbering, a substitution at position 235 according to EU numbering, or both (e.g., a Leu to Ala substitution at position 234 and/or a Leu to Ala substitution at position 235).

The anti-PD-1 antibody molecule, or a pharmaceutically acceptable salt thereof, of the present disclosure also includes, for example, a heavy chain variable domain and a constant region, a light chain variable domain and a constant region, or both, comprising the amino acid sequence of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as shown in Table 4; or a sequence substantially identical thereto.

The anti-PD-1 antibody molecule, or a pharmaceutically acceptable salt thereof, according to the present disclosure includes at least one, two, or three complementarity determining regions (CDRs) from a heavy chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecule, or a pharmaceutically acceptable salt thereof, according to the present disclosure includes, for example, at least one, two, or three CDRs (or collectively all of the CDRs) from a heavy chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, according to the present disclosure includes, for example, at least one, two, or three CDRs from a light chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequence.

The anti-PD-1 antibody molecule, or a pharmaceutically acceptable salt thereof, includes, for example, at least one, two, or three CDRs (or collectively all of the CDRs) from a heavy chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, includes, for example, at least one, two, or three CDRs from a light chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequence.

The anti-PD-1 antibody molecule, or a pharmaceutically acceptable salt thereof, includes, for example, at least one, two, or three CDRs (or collectively all of the CDRs) from a light chain variable region comprising an amino acid sequence shown in Table 1, or encoded by a nucleotide sequence shown in Table 1. One or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1. In certain embodiments, the anti-PD-1 antibody molecule, or a pharmaceutically acceptable salt thereof, includes a substitution in a light chain CDR, e.g., one or more substitutions in a CDR1, CDR2 and/or CDR3 of the light chain. The anti-PD-1 antibody molecule, or a pharmaceutically acceptable salt thereof, includes a substitution in the light chain CDR3 at position 102 of the light variable region, e.g., a substitution of a cysteine to tyrosine, or a cysteine to serine residue, at position 102 of the light variable region according to Table 1 (e.g., SEQ ID NO: 54 or 70 for a modified sequence).

The anti-PD-1 antibody molecule, or a pharmaceutically acceptable salt thereof, according to the present disclosure includes, for example, at least one, two, three, four, five or six CDRs (or collectively all of the CDRs) from a heavy and light chain variable region comprising an amino acid sequence shown in Table 1, or encoded by a nucleotide sequence shown in Table 1. In one embodiment, one or more of the CDRs (or collectively all of the CDRs) have one, two, three, four, five, six or more changes, e.g., amino acid substitutions or deletions, relative to the amino acid sequence shown in Table 1, or encoded by a nucleotide sequence shown in Table 1.

The anti-PD-1 antibody molecule, or a pharmaceutically acceptable salt thereof, of the present disclosure includes, for example, all six CDRs from an antibody described herein, e.g., an antibody chosen from any of BAP049-Clone-B or BAP049-Clone-E; or as described in Table 1, or encoded by the nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). The anti-PD-1 antibody molecule, or a pharmaceutically acceptable salt thereof, may also include any CDR described herein. The anti-PD-1 antibody molecule, or a pharmaceutically acceptable salt thereof, includes a substitution in a light chain CDR, e.g., one or more substitutions in a CDR1, CDR2 and/or CDR3 of the light chain. The anti-PD-1 antibody molecule, or a pharmaceutically acceptable salt thereof, according to the present disclosure, includes a substitution in the light chain CDR3 at position 102 of the light variable region, e.g., a substitution of a cysteine to tyrosine, or a cysteine to serine residue, at position 102 of the light variable region according to Table 1.

The anti-PD-1 antibody molecule, or a pharmaceutically acceptable salt thereof, of the present disclosure, includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

The anti-PD-1 antibody molecule, or a pharmaceutically acceptable salt thereof, of the present invention, includes, for example, at least one, two, or three CDRs according to Kabat et al. (e.g., at least one, two, or three CDRs according to the Kabat definition as set out in Table 1) from a light chain variable region of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

The anti-PD-1 antibody molecule, or a pharmaceutically acceptable salt thereof, according to the present disclosure, includes, for example, at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

The anti-PD-1 antibody molecule, or a pharmaceutically acceptable salt thereof, of the present disclosure, includes all six CDRs according to Kabat et al. (e.g., all six CDRs according to the Kabat definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. The anti-PD-1 antibody molecule, or a pharmaceutically acceptable salt thereof, according to the present disclosure, may include any CDR described herein.

The anti-PD-1 antibody molecule, or a pharmaceutically acceptable salt thereof, according to the present disclosure, includes, for example at least one, two, or three Chothia hypervariable loops (e.g., at least one, two, or three hypervariable loops according to the Chothia definition as set out in Table 1) from a heavy chain variable region of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

The anti-PD-1 antibody molecule, or a pharmaceutically acceptable salt thereof, according to the present disclosure, includes, for example, at least one, two, or three Chothia hypervariable loops (e.g., at least one, two, or three hypervariable loops according to the Chothia definition as set out in Table 1) of a light chain variable region of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

The anti-PD-1 antibody molecule, or a pharmaceutically acceptable salt thereof, according to the present disclosure, includes, for example, at least one, two, three, four, five, or six hypervariable loops (e.g., at least one, two, three, four, five, or six hypervariable loops according to the Chothia definition as set out in Table 1) from the heavy and light chain variable regions of an antibody described herein, e.g., an antibody chosen from any of BAP049-Clone-B or BAP049-Clone-E; or as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 1.

The anti-PD-1 antibody molecule, or a pharmaceutically acceptable salt thereof, according to the present disclosure, includes, for example, all six hypervariable loops (e.g., all six hypervariable loops according to the Chothia definition as set out in Table 1) of an antibody described herein, e.g., an antibody chosen from any of BAP049-Clone-B or BAP049-Clone-E, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 1. The anti-PD-1 antibody molecule, or a pharmaceutically acceptable salt thereof, according to the present disclosure may include any hypervariable loop described herein.

The anti-PD-1 antibody molecule, or a pharmaceutically acceptable salt thereof, according to the present disclosure, includes, for example, at least one, two, or three hypervariable loops that have the same canonical structures as the corresponding hypervariable loop of an antibody described herein, e.g., an antibody chosen from any of BAP049-Clone-B or BAP049-Clone-E, e.g., the same canonical structures as at least loop 1 and/or loop 2 of the heavy and/or light chain variable domains of an antibody described herein. (See, e.g., Chothia et al. J. Mol. Biol. 1992, 227, 799; Tomlinson et al. J. Mol. Biol. 1992, 227:776-798 for descriptions of hypervariable loop canonical structures). These structures can be determined by inspection of the tables described in these references.

The anti-PD-1 antibody molecule, or a pharmaceutically acceptable salt thereof, according to the present disclosure, may also include, for example, a combination of CDRs or hypervariable loops defined according to the Kabat et al. and Chothia et al. as described herein in Table 1.

The anti-PD-1 antibody molecule, or a pharmaceutically acceptable salt thereof, according to the present disclosure, includes, for example, at least one, two or three CDRs or hypervariable loops from a heavy chain variable region of an antibody described herein, e.g., an antibody chosen from any of BAP049-Clone-B or BAP049-Clone-E, according to the Kabat and Chothia definition (e.g., at least one, two, or three CDRs or hypervariable loops according to the Kabat and Chothia definition as set out in Table 1); or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 1.

For example, the anti-PD-1 antibody molecule, or a pharmaceutically acceptable salt thereof, according to the present disclosure, can include VH CDR1 according to Kabat et al. or VH hypervariable loop 1 according to Chothia et al., or a combination thereof, e.g., as shown in Table 1. The combination of Kabat and Chothia CDR of VH CDR1 comprises the amino acid sequence GYTFTTYWMH (SEQ ID NO: 224), or an amino acid sequence substantially identical thereto (e.g., having at least one amino acid alteration, but not more than two, three or four alterations (e.g., substitutions, deletions, or insertions, e.g., conservative substitutions). The anti-PD-1 antibody molecule can further include, e.g., VH CDRs 2-3 according to Kabat et al. and VL CDRs 1-3 according to Kabat et al., e.g., as shown in Table 1. Accordingly, the framework regions (FW) are defined based on a combination of CDRs defined according to Kabat et al. and hypervariable loops defined according to Chothia et al. For example, the anti-PD-1 antibody molecule can include VH FW1 defined based on VH hypervariable loop 1 according to Chothia et al. and VH FW2 defined based on VH CDRs 1-2 according to Kabat et al., e.g., as shown in Table 1. The anti-PD-1 antibody molecule can further include, e.g., VH FWs 3-4 defined based on VH CDRs 2-3 according to Kabat et al. and VL FWs 1-4 defined based on VL CDRs 1-3 according to Kabat et al.

The anti-PD-1 antibody molecule, or a pharmaceutically acceptable salt thereof, according to the present disclosure, includes at least one, two or three CDRs from a light chain variable region of an antibody described herein, e.g., an antibody chosen from any of BAP049-Clone-B or BAP049-Clone-E, according to the Kabat and Chothia definitions (e.g., at least one, two, or three CDRs according to the Kabat and Chothia definitions as set out in Table 1).

The anti-PD-1 antibody molecule, or a pharmaceutically acceptable salt thereof, according to the present disclosure, includes:

(a) a heavy chain variable region (VH) comprising a VHCDR1 amino acid sequence of SEQ ID NO: 4, a VHCDR2 amino acid sequence of SEQ ID NO: 5, and a VHCDR3 amino acid sequence of SEQ ID NO: 3; and a light chain variable region (VL) comprising a VLCDR1 amino acid sequence of SEQ ID NO: 13, a VLCDR2 amino acid sequence of SEQ ID NO: 14, and a VLCDR3 amino acid sequence of SEQ ID NO: 33;
(b) a VH comprising a VHCDR1 amino acid sequence chosen from SEQ ID NO: 1; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32;
(c) a VH comprising a VHCDR1 amino acid sequence of SEQ ID NO: 224, a VHCDR2 amino acid sequence of SEQ ID NO: 5, and a VHCDR3 amino acid sequence of SEQ ID NO: 3; and a VL comprising a VLCDR1 amino acid sequence of SEQ ID NO: 13, a VLCDR2 amino acid sequence of SEQ ID NO: 14, and a VLCDR3 amino acid sequence of SEQ ID NO: 33; or
(d) a VH comprising a VHCDR1 amino acid sequence of SEQ ID NO: 224; a VHCDR2 amino acid sequence of SEQ ID NO: 2; and a VHCDR3 amino acid sequence of SEQ ID NO: 3; and a VL comprising a VLCDR1 amino acid sequence of SEQ ID NO: 10, a VLCDR2 amino acid sequence of SEQ ID NO: 11, and a VLCDR3 amino acid sequence of SEQ ID NO: 32.

The anti-PD-1 antibody molecule, or a pharmaceutically acceptable salt thereof, according to the present disclosure, comprises a heavy chain variable region (VH) comprising a VHCDR1 amino acid sequence chosen from SEQ ID NO: 1, SEQ ID NO: 4, or SEQ ID NO: 224; a VHCDR2 amino acid sequence of SEQ ID NO: 2 or SEQ ID NO: 5; and a VHCDR3 amino acid sequence of SEQ ID NO: 3; and a light chain variable region (VL) comprising a VLCDR1 amino acid sequence of SEQ ID NO: 10 or SEQ ID NO: 13, a VLCDR2 amino acid sequence of SEQ ID NO: 11 or SEQ ID NO: 14, and a VLCDR3 amino acid sequence of SEQ ID NO: 32 or SEQ ID NO: 33.

The anti-PD-1 antibody molecule, or a pharmaceutically acceptable salt thereof, according to the present disclosure, can comprise, for example, a heavy chain variable domain comprising the amino acid sequence of SEQ ID NO: 38 and a light chain variable domain comprising the amino acid sequence of SEQ ID NO: 70.

The anti-PD-1 antibody molecule, or a pharmaceutically acceptable salt thereof, according to the present disclosure, can comprise, for example, a heavy chain comprising the amino acid sequence of SEQ ID NO: 91 and a light chain comprising the amino acid sequence of SEQ ID NO: 72.

The anti-PD-1 antibody molecule, or a pharmaceutically acceptable salt thereof, according to the present disclosure, comprises a heavy chain variable region (VH) comprising a HCDR1, a HCDR2 and a HCDR3 amino acid sequence of BAP049-Clone-B or BAP049-Clone-E as described in Table 1 and a light chain variable region (VL) comprising a LCDR1, a LCDR2 and a LCDR3 amino acid sequence of BAP049-Clone-B or BAP049-Clone-E as described in Table 1.

The anti-PD-1 antibody molecule, or a pharmaceutically acceptable salt thereof, according to the present disclosure, comprises a heavy chain variable region (VH) comprising a HCDR1, a HCDR2 and a HCDR3 amino acid sequence of BAP049-Clone-E as described in Table 1 and a light chain variable region (VL) comprising a LCDR1, a LCDR2 and a LCDR3 amino acid sequence of BAP049-Clone-E as described in Table 1.

It is understood that the anti-PD-1 antibody molecule, or the anti-PD-1 antibody molecule, of the present disclosure may have additional conservative or non-essential amino acid substitutions, which do not have a substantial effect on their functions.

The term “antibody molecule” refers to a protein, e.g., an immunoglobulin chain or fragment thereof, comprising at least one immunoglobulin variable domain sequence. The term “antibody molecule” includes, for example, a monoclonal antibody (including a full length antibody which has an immunoglobulin Fc region). An antibody molecule comprises a full length antibody, or a full length immunoglobulin chain, or an antigen binding or functional fragment of a full length antibody, or a full length immunoglobulin chain. An antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibodies of the present disclosure can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. An antibody molecule can also be a multi-specific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope.

The term “Pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compound herein is to be understood as referring also to the corresponding salt, as appropriate and expedient. The salts of the inhibitors, as described herein, are preferably pharmaceutically acceptable salts; suitable counter-ions forming pharmaceutically acceptable salts are known in the field.

The term “pharmaceutically acceptable” refers to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

The term “inhibition” or “inhibitor” includes a reduction in a certain parameter, e.g., an activity, of a given molecule, e.g., an immune checkpoint inhibitor, such as the anti-PD-1 antibody molecule. For example, inhibition of an activity, e.g., a PD-1 or PD-L1 activity, of at least 5%, 10%, 20%, 30%, 40% or more is included by this term. Thus, inhibition need not be 100%.

The term “cancer” refers to a disease characterized by the rapid and uncontrolled growth of aberrant cell proliferation. Cancer cells can spread locally or through the bloodstream and lymphatic system to other parts of the body. Examples of various cancers are, but are not limited to, lymphoma, ovarian cancer, non-small cell lung cancer (NSCLC), cervical cancer, gastric cancer, breast cancer, colorectal cancer, or melanoma. The cancer can be advanced cancer.

According to the present disclosure the particularly amenable disease condition to be treated with the aforementioned combination is NSCLC. Preferably, the cancer is characterized as having an anaplastic lymphoma kinase (ALK) rearrangement or translocation (i.e. ALK-positive), e.g., an ALK fusion, e.g., an EML4-ALK fusion. The present combination is particularly suitable for the treatment of patients with metastatic, ALK-positive NSCLC.

The term “treatment” comprises, for example, the therapeutic administration of the combination of ceritinib, or a pharmaceutically acceptable salt thereof, and an anti-PD-1 antibody molecule, or a pharmaceutically acceptable salt thereof, as described herein to a warm-blooded animal, in particular a human being, in need of such treatment with the aim to cure the disease or to have an effect on disease regression or on the delay of progression of a disease. The terms “treat”, “treating” or “treatment” of any disease or disorder refers to ameliorating the disease or disorder (e.g. slowing or arresting or reducing the development of the disease or at least one of the clinical symptoms thereof), to preventing or delaying the onset or development or progression of the disease or disorder.

The anti-PD-1 antibody molecule can be given that they are antibody molecules, be administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the anti-PD-1 antibody molecule is administered at a dose from about 2 to 20 mg/kg every other week. In the event that new viable and effective ways, such as nasally, orally by inhalation or otherwise, are found to be suitable to effectively deliver antibodies systemically, the antibodies mentioned herein could be administered via those routes as well. In such case, the dose would need to be adapted to the dose that is bioequivalent to the suitable subcutaneous or intravenous dose.

More specifically, Nivolumab can be administered intravenously at a dose from about 1 mg/kg to 3 mg/kg, e.g., about 1 mg/kg, 2 mg/kg or 3 mg/kg, every two weeks. Particularly the anti-PD-1 antibody molecule Nivolumab is administered intravenously at a dose of about 3 mg/kg at 2-week intervals. Patients receiving nivolumab in combination with ceritinib can have nivolumab administered at a dose of 3 mg/kg as a 60 (+/−10) minute intravenous infusion every two weeks (i.e. Q2W).

The PDR-001, or a pharmaceutically acceptable salt thereof, as described herein, for use in the treatment of cancer, is administered by injection (e.g. subcutaneously or intravenously) at a dose of 300-400 mg/day. Preferably, the PDR-001, or a pharmaceutically acceptable salt thereof, is administered intravenously in a single dose of 300 to 400 mg/day. Most preferably, the PDR-001, or a pharmaceutically acceptable salt thereof, is administered in a single dose of 400 mg/day. Most preferably, the anti-PD-1 antibody molecule PDR-001 is administered at a dose of 400 mg/kg every four weeks. The dose can be administered in a single bolus or in several divided doses.

The methods and compositions described herein can be used in combination with further agents or therapeutic modalities. After the run-in period (i.e. 2 to 3 cycles) the combination therapies can be administered simultaneously or sequentially in any order. Any combination and sequence of the anti-PD-1 or PD-L1 antibody molecules and other therapeutic agents, procedures or modalities (e.g., as described herein) can be used. However, best result can be achieved if the drugs forming the combination are administered as explained herein (e.g. in relation to the sequence of administration, or intake of food). The combination therapies can be administered during periods of active disorder, or during a period of remission or less active disease. The combination therapies can be administered before the other treatment, concurrently with the treatment, post-treatment, or during remission of the disorder.

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 300 to 400 mg/day. For intravenous injection or infusion, therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients as required, followed by filtered sterilization. Generally, dispersions are prepared by incorporating the active compound into a sterile vehicle that contains a basic dispersion medium and the required other ingredients. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

It would be understood that the route and/or mode of administration will vary depending upon the desired results. For example, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (e.g., Sustained and Controlled Release Drug Delivery Systems, J. R. Robinson, ed., Marcel Dekker, Inc., New York, 1978).

When used for the manufacture of a medicament for the treatment of cancer or in a method of treating a cancer in a patient in need thereof, (i) and (ii) can be used in doses and dosing schedules as explained above.

By the same token, the present disclosure also provides a method for the treatment of cancer, comprising administering an effective amount of the combination partners (i) ceritinib, or a pharmaceutically acceptable salt thereof and anti-PD-1 antibody molecule (ii), or a pharmaceutically acceptable salt thereof) to a patient in need thereof.

The combination partners (i) and (ii), as described herein, can be synergistically active, while causing less side effects caused by the combination, such as rash.

The term “effective amount” or “therapeutically effective amount” of the combination partners of the present disclosure, refers to an amount effective, at dosages and for periods of time necessary, to achieve the desired therapeutic result. A therapeutically effective amount of the combination partners may vary according to factors such as the disease state, age, sex, and weight of the individual. A therapeutically effective amount is also one in which any toxic or detrimental effects of the combination, as described herein, is outweighed by the therapeutically beneficial effects. A “therapeutically effective dosage” preferably inhibits a measurable parameter, e.g., tumor growth rate by at least about 20%, more preferably by at least about 40%, even more preferably by at least about 60%, and still more preferably by at least about 80% relative to untreated subjects.

TABLE 1
Amino acid and nucleotide sequences for humanized antibody molecules, which
can be used as anti-PD-1 antibody molecule according to the present
dislcosure. The antibody molecules include BAP049-Clone-B and BAP049-Clone-
E. The amino acid and nucleotide sequences of the heavy and light chain
CDRs, the heavy and light chain variable regions, and the heavy and light
chains are shown.
BAP049-Clone-B HC

| | | |
|---|---|---|
| SEQ ID NO: 1 (Kabat) | HCDR1 | TYWMH |
| SEQ ID NO: 2 (Kabat) | HCDR2 | NIYPGTGGSNFDEKFKN |
| SEQ ID NO: 3 (Kabat) | HCDR3 | WTTGTGAY |
| SEQ ID NO: 4 (Chothia) | HCDR1 | GYTFTTY |
| SEQ ID NO: 5 (Chothia) | HCDR2 | YPGTGG |
| SEQ ID NO: 3 (Chothia) | HCDR3 | WTTGTGAY |
| SEQ ID NO: 38 | VH | EVQLVQSGAEVKKPGESLRISCKGSGYTFT |
| | | TYWMHWVRQATGQGLEWMGNIYPGTGGS |
| | | NFDEKFKNRVTITADKSTSTAYMELSSLRSE |
| | | DTAVYYCTRVVTTGTGAYWGQGTTVTVSS |
| SEQ ID NO: 95 | DNA VH | GAGGTGCAGCTGGTGCAGTCAGGCGCCG |
| | | AAGTGAAGAAGCCCGGCGAGTCACTGAG |
| | | AATTAGCTGTAAAGGTTCAGGCTACACCT |
| | | TCACTACCTACTGGATGCACTGGGTCCGC |
| | | CAGGCTACCGGTCAAGGCCTCGAGTGGA |
| | | TGGGTAATATCTACCCCGGCACCGGCGG |
| | | CTCTAACTTCGACGAGAAGTTTAAGAATA |
| | | GAGTGACTATCACCGCCGATAAGTCTACT |
| | | AGCACCGCCTATATGGAACTGTCTAGCCT |
| | | GAGATCAGAGGACACCGCCGTCTACTACT |
| | | GCACTAGGTGGACTACCGGCACAGGCGC |
| | | CTACTGGGGTCAAGGCACTACCGTGACC |
| | | GTGTCTAGC |
| SEQ ID NO: 91 | HC | EVQLVQSGAEVKKPGESLRISCKGSGYTFT |
| | | TYWMHWVRQATGQGLEWMGNIYPGTGGS |
| | | NFDEKFKNRVTITADKSTSTAYMELSSLRSE |
| | | DTAVYYCTRVVTTGTGAYWGQGTTVTVSSA |
| | | STKGPSVFPLAPCSRSTSESTAALGCLVKD |
| | | YFPEPVTVSWNSGALTSGVHTFPAVLQSSG |
| | | LYSLSSVVTVPSSSLGTKTYTCNVDHKPSN |
| | | TKVDKRVESKYGPPCPPCPAPEFLGGPSVF |
| | | LFPPKPKDTLMISRTPEVTCVVVDVSQEDP |
| | | EVQFNWYVDGVEVHNAKTKPREEQFNSTY |
| | | RVVSVLTVLHQDWLNGKEYKCKVSNKGLP |
| | | SSIEKTISKAKGQPREPQVYTLPPSQEEMTK |
| | | NQVSLTCLVKGFYPSDIAVEWESNGQPENN |
| | | YKTTPPVLDSDGSFFLYSRLTVDKSRWQEG |
| | | NVFSCSVMHEALHNHYTQKSLSLSLG |
| SEQ ID NO: 96 | DNA HC | GAGGTGCAGCTGGTGCAGTCAGGCGCCG |
| | | AAGTGAAGAAGCCCGGCGAGTCACTGAG |
| | | AATTAGCTGTAAAGGTTCAGGCTACACCT |
| | | TCACTACCTACTGGATGCACTGGGTCCGC |
| | | CAGGCTACCGGTCAAGGCCTCGAGTGGA |
| | | TGGGTAATATCTACCCCGGCACCGGCGG |
| | | CTCTAACTTCGACGAGAAGTTTAAGAATA |
| | | GAGTGACTATCACCGCCGATAAGTCTACT |
| | | AGCACCGCCTATATGGAACTGTCTAGCCT |
| | | GAGATCAGAGGACACCGCCGTCTACTACT |
| | | GCACTAGGTGGACTACCGGCACAGGCGC |
| | | CTACTGGGGTCAAGGCACTACCGTGACC |
| | | GTGTCTAGCGCTAGCACTAAGGGCCCGT |
| | | CCGTGTTCCCCCTGGCACCTTGTAGCCG |
| | | GAGCACTAGCGAATCCACCGCTGCCCTC |
| | | GGCTGCCTGGTCAAGGATTACTTCCCGG |
| | | AGCCCGTGACCGTGTCCTGGAACAGCGG |
| | | AGCCCTGACCTCCGGAGTGCACACCTTC |
| | | CCCGCTGTGCTGCAGAGCTCCGGGCTGT |
| | | ACTCGCTGTCGTCGGTGGTCACGGTGCC |
| | | TTCATCTAGCCTGGGTACCAAGACCTACA |
| | | CTTGCAACGTGGACCACAAGCCTTCCAAC |
| | | ACTAAGGTGGACAAGCGCGTCGAATCGA |
| | | AGTACGGCCCACCGTGCCCGCCTTGTCC |
| | | CGCGCCGGAGTTCCTCGGCGGTCCCTCG |
| | | GTCTTTCTGTTCCCACCGAAGCCCAAGGA |
| | | CACTTTGATGATTTCCCGCACCCCTGAAG |
| | | TGACATGCGTGGTCGTGGACGTGTCACA |
| | | GGAAGATCCGGAGGTGCAGTTCAATTGG |
| | | TACGTGGATGGCGTCGAGGTGCACAACG |
| | | CCAAAACCAAGCCGAGGGAGGAGCAGTT |
| | | CAACTCCACTTACCGCGTCGTGTCCGTGC |
| | | TGACGGTGCTGCATCAGGACTGGCTGAA |
| | | CGGGAAGGAGTACAAGTGCAAAGTGTCC |
| | | AACAAGGGACTTCCTAGCTCAATCGAAAA |
| | | GACCATCTCGAAAGCCAAGGGACAGCCC |
| | | CGGGAACCCCAAGTGTATACCCTGCCAC |
| | | CGAGCCAGGAAGAAATGACTAAGAACCAA |
| | | GTCTCATTGACTTGCCTTGTGAAGGGCTT |
| | | CTACCCATCGGATATCGCCGTGGAATGG |
| | | GAGTCCAACGGCCAGCCGGAAAACAACT |
| | | ACAAGACCACCCCTCCGGTGCTGGACTC |
| | | AGACGGATCCTTCTTCCTCTACTCGCGGC |
| | | TGACCGTGGATAAGAGCAGATGGCAGGA |
| | | GGGAAATGTGTTCAGCTGTTCTGTGATGC |
| | | ATGAAGCCCTGCACAACCACTACACTCAG |
| | | AAGTCCCTGTCCCTCTCCCTGGGA |

BAP049-Clone-B LC

| | | |
|---|---|---|
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 54 | VL | EIVLTQSPATLSLSPGERATLSCKSSQSLLD |
| | | SGNQKNFLTWYQQKPGKAPKLLIYWASTR |
| | | ESGVPSRFSGSGSGTDFTFTISSLQPEDIAT |
| | | YYCQNDYSYPYTFGQGTKVEIK |
| SEQ ID NO: 97 | DNA VL | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTAAA |
| | | GCCCCTAAGCTGCTGATCTACTGGGCCTC |
| | | TACTAGAGAATCAGGCGTGCCCTCTAGGT |
| | | TTAGCGGTAGCGGTAGTGGCACCGACTT |
| | | CACCTTCACTATCTCTAGCCTGCAGCCCG |
| | | AGGATATCGCTACCTACTACTGTCAGAAC |
| | | GACTATAGCTACCCCTACACCTTCGGTCA |
| | | AGGCACTAAGGTCGAGATTAAG |
| SEQ ID NO: 56 | LC | EIVLTQSPATLSLSPGERATLSCKSSQSLLD |
| | | SGNQKNFLTWYQQKPGKAPKLLIYWASTR |
| | | ESGVPSRFSGSGSGTDFTFTISSLQPEDIAT |
| | | YYCQNDYSYPYTFGQGTKVEIKRTVAAPSV |
| | | FIFPPSDEQLKSGTASVVCLLNNFYPREAKV |
| | | QWKVDNALQSGNSQESVTEQDSKDSTYSL |
| | | SSTLTLSKADYEKHKVYACEVTHQGLSSPV |
| | | TKSFNRGEC |
| SEQ ID NO: 98 | DNA LC | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTAAA |
| | | GCCCCTAAGCTGCTGATCTACTGGGCCTC |
| | | TACTAGAGAATCAGGCGTGCCCTCTAGGT |
| | | TTAGCGGTAGCGGTAGTGGCACCGACTT |
| | | CACCTTCACTATCTCTAGCCTGCAGCCCG |
| | | AGGATATCGCTACCTACTACTGTCAGAAC |
| | | GACTATAGCTACCCCTACACCTTCGGTCA |
| | | AGGCACTAAGGTCGAGATTAAGCGTACG |
| | | GTGGCCGCTCCCAGCGTGTTCATCTTCCC |
| | | CCCCAGCGACGAGCAGCTGAAGAGCGGC |
| | | ACCGCCAGCGTGGTGTGCCTGCTGAACA |
| | | ACTTCTACCCCCGGGAGGCCAAGGTGCA |
| | | GTGGAAGGTGGACAACGCCCTGCAGAGC |
| | | GGCAACAGCCAGGAGAGCGTCACCGAGC |
| | | AGGACAGCAAGGACTCCACCTACAGCCT |
| | | GAGCAGCACCCTGACCCTGAGCAAGGCC |
| | | GACTACGAGAAGCATAAGGTGTACGCCT |
| | | GCGAGGTGACCCACCAGGGCCTGTCCAG |
| | | CCCCGTGACCAAGAGCTTCAACAGGGGC |
| | | GAGTGC |
| SEQ ID NO: 92 | DNA HC | GAAGTGCAGCTGGTGCAGTCTGGCGCCG |
| | | AAGTGAAGAAGCCTGGCGAGTCCCTGCG |
| | | GATCTCCTGCAAGGGCTCTGGCTACACCT |
| | | TCACCACCTACTGGATGCACTGGGTGCG |
| | | ACAGGCTACCGGCCAGGGCCTGGAATGG |
| | | ATGGGCAACATCTATCCTGGCACCGGCG |
| | | GCTCCAACTTCGACGAGAAGTTCAAGAAC |
| | | AGAGTGACCATCACCGCCGACAAGTCCA |
| | | CCTCCACCGCCTACATGGAACTGTCCTCC |
| | | CTGAGATCCGAGGACACCGCCGTGTACT |
| | | ACTGCACCCGGTGGACAACCGGCACAGG |
| | | CGCTTATTGGGGCCAGGGCACCACAGTG |
| | | ACCGTGTCCTCTGCTTCTACCAAGGGGCC |
| | | CAGCGTGTTCCCCCTGGCCCCCTGCTCC |
| | | AGAAGCACCAGCGAGAGCACAGCCGCCC |
| | | TGGGCTGCCTGGTGAAGGACTACTTCCC |
| | | CGAGCCCGTGACCGTGTCCTGGAACAGC |
| | | GGAGCCCTGACCAGCGGCGTGCACACCT |
| | | TCCCCGCCGTGCTGCAGAGCAGCGGCCT |
| | | GTACAGCCTGAGCAGCGTGGTGACCGTG |
| | | CCCAGCAGCAGCCTGGGCACCAAGACCT |
| | | ACACCTGTAACGTGGACCACAAGCCCAG |
| | | CAACACCAAGGTGGACAAGAGGGTGGAG |
| | | AGCAAGTACGGCCCACCCTGCCCCCCCT |
| | | GCCCAGCCCCCGAGTTCCTGGGCGGACC |
| | | CAGCGTGTTCCTGTTCCCCCCCAAGCCCA |
| | | AGGACACCCTGATGATCAGCAGAACCCC |
| | | CGAGGTGACCTGTGTGGTGGTGGACGTG |
| | | TCCCAGGAGGACCCCGAGGTCCAGTTCA |
| | | ACTGGTACGTGGACGGCGTGGAGGTGCA |
| | | CAACGCCAAGACCAAGCCCAGAGAGGAG |
| | | CAGTTTAACAGCACCTACCGGGTGGTGTC |
| | | CGTGCTGACCGTGCTGCACCAGGACTGG |
| | | CTGAACGGCAAAGAGTACAAGTGTAAGGT |
| | | CTCCAACAAGGGCCTGCCAAGCAGCATC |
| | | GAAAAGACCATCAGCAAGGCCAAGGGCC |
| | | AGCCTAGAGAGCCCCAGGTCTACACCCT |
| | | GCCACCCAGCCAAGAGGAGATGACCAAG |
| | | AACCAGGTGTCCCTGACCTGTCTGGTGAA |
| | | GGGCTTCTACCCAAGCGACATCGCCGTG |
| | | GAGTGGGAGAGCAACGGCCAGCCCGAGA |
| | | ACAACTACAAGACCACCCCCCCAGTGCTG |
| | | GACAGCGACGGCAGCTTCTTCCTGTACA |
| | | GCAGGCTGACCGTGGACAAGTCCAGATG |
| | | GCAGGAGGGCAACGTCTTTAGCTGCTCC |
| | | GTGATGCACGAGGCCCTGCACAACCACT |
| | | ACACCCAGAAGAGCCTGAGCCTGTCCCT |
| | | GGGC |

BAP049-Clone-E LC

| | | |
|---|---|---|
| SEQ ID NO: 10 (Kabat) | LCDR1 | KSSQSLLDSGNQKNFLT |
| SEQ ID NO: 11 (Kabat) | LCDR2 | WASTRES |
| SEQ ID NO: 32 (Kabat) | LCDR3 | QNDYSYPYT |
| SEQ ID NO: 13 (Chothia) | LCDR1 | SQSLLDSGNQKNF |
| SEQ ID NO: 14 (Chothia) | LCDR2 | WAS |
| SEQ ID NO: 33 (Chothia) | LCDR3 | DYSYPY |
| SEQ ID NO: 70 | VL | EIVLTQSPATLSLSPGERATLSCKSSQSLLD |
| | | SGNQKNFLTWYQQKPGQAPRLLIYWASTR |
| | | ESGVPSRFSGSGSGTDFTFTISSLEAEDAA |
| | | TYYCQNDYSYPYTFGQGTKVEIK |
| SEQ ID NO: 106 | DNA VL | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTCA |
| | | AGCCCCTAGACTGCTGATCTACTGGGCCT |
| | | CTACTAGAGAATCAGGCGTGCCCTCTAGG |
| | | TTTAGCGGTAGCGGTAGTGGCACCGACTT |
| | | CACCTTCACTATCTCTAGCCTGGAAGCCG |
| | | AGGACGCCGCTACCTACTACTGTCAGAAC |
| | | GACTATAGCTACCCCTACACCTTCGGTCA |
| | | AGGCACTAAGGTCGAGATTAAG |
| SEQ ID NO: 72 | LC | EIVLTQSPATLSLSPGERATLSCKSSQSLLD |
| | | SGNQKNFLTWYQQKPGQAPRLLIYWASTR |
| | | ESGVPSRFSGSGSGTDFTFTISSLEAEDAA |
| | | TYYCQNDYSYPYTFGQGTKVEIKRTVAAPS |
| | | VFIFPPSDEQLKSGTASVVCLLNNFYPREAK |
| | | VQWKVDNALQSGNSQESVTEQDSKDSTYS |
| | | LSSTLTLSKADYEKHKVYACEVTHQGLSSP |
| | | VTKSFNRGEC |
| SEQ ID NO: 107 | DNA LC | GAGATCGTCCTGACTCAGTCACCCGCTAC |
| | | CCTGAGCCTGAGCCCTGGCGAGCGGGCT |
| | | ACACTGAGCTGTAAATCTAGTCAGTCACT |
| | | GCTGGATAGCGGTAATCAGAAGAACTTCC |
| | | TGACCTGGTATCAGCAGAAGCCCGGTCA |
| | | AGCCCCTAGACTGCTGATCTACTGGGCCT |
| | | CTACTAGAGAATCAGGCGTGCCCTCTAGG |
| | | TTTAGCGGTAGCGGTAGTGGCACCGACTT |
| | | CACCTTCACTATCTCTAGCCTGGAAGCCG |
| | | AGGACGCCGCTACCTACTACTGTCAGAAC |
| | | GACTATAGCTACCCCTACACCTTCGGTCA |
| | | AGGCACTAAGGTCGAGATTAAGCGTACG |
| | | GTGGCCGCTCCCAGCGTGTTCATCTTCCC |
| | | CCCCAGCGACGAGCAGCTGAAGAGCGGC |
| | | ACCGCCAGCGTGGTGTGCCTGCTGAACA |
| | | ACTTCTACCCCCGGGAGGCCAAGGTGCA |
| | | GTGGAAGGTGGACAACGCCCTGCAGAGC |
| | | GGCAACAGCCAGGAGAGCGTCACCGAGC |
| | | AGGACAGCAAGGACTCCACCTACAGCCT |
| | | GAGCAGCACCCTGACCCTGAGCAAGGCC |
| | | GACTACGAGAAGCATAAGGTGTACGCCT |
| | | GCGAGGTGACCCACCAGGGCCTGTCCAG |
| | | CCCCGTGACCAAGAGCTTCAACAGGGGC |
| | | GAGTGC |

BAP049-Clone-B HC

| | | |
|---|---|---|
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAA |
| | | CTTCGACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |

BAP049-Clone-B LC

| | | |
|---|---|---|
| SEQ ID NO: 138 (Kabat) | LCDR1 | AAATCTAGTCAGTCACTGCTGGATAGCGG |
| | | TAATCAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCA |
| | | GAAGAACTTC |
| SEQ ID NO: 142 (Chothia) | LCDR2 | TGGGCCTCT |
| SEQ ID NO: 143 (Chothia) | LCDR3 | GACTATAGCTACCCCTAC |

BAP049-Clone-E HC

| | | |
|---|---|---|
| SEQ ID NO: 133 (Kabat) | HCDR1 | ACCTACTGGATGCAC |
| SEQ ID NO: 134 (Kabat) | HCDR2 | AATATCTACCCCGGCACCGGCGGCTCTAA |
| | | CTTCGACGAGAAGTTTAAGAAT |
| SEQ ID NO: 135 (Kabat) | HCDR3 | TGGACTACCGGCACAGGCGCCTAC |
| SEQ ID NO: 136 (Chothia) | HCDR1 | GGCTACACCTTCACTACCTAC |
| SEQ ID NO: 137 (Chothia) | HCDR2 | TACCCCGGCACCGGCGGC |
| SEQ ID NO: 135 (Chothia) | HCDR3 | TGGACTACCGGCACAGGCGCCTAC |

BAP049-Clone-E LC

| | | |
|---|---|---|
| SEQ ID NO: 138 (Kabat) | LCDR1 | AAATCTAGTCAGTCACTGCTGGATAGCGG |
| | | TAATCAGAAGAACTTCCTGACC |
| SEQ ID NO: 139 (Kabat) | LCDR2 | TGGGCCTCTACTAGAGAATCA |
| SEQ ID NO: 140 (Kabat) | LCDR3 | CAGAACGACTATAGCTACCCCTACACC |
| SEQ ID NO: 141 (Chothia) | LCDR1 | AGTCAGTCACTGCTGGATAGCGGTAATCA |
| | | GAAGAACTTC |
| SEQ ID NO: 142 (Chothia) | LCDR2 | TGGGCCTCT |
| SEQ ID NO: 143 (Chothia) | LCDR3 | GACTATAGCTACCCCTAC |

TABLE 2
Amino acid and nucleotide sequences of the heavy and light chain
framework regions for humanized mAbs BAP049-Clone-B and BAP049-Clone-E

| | Amino Acid Sequence | Nucleotide Sequence |
|---|---|---|
| VHFW1 | EVQLVQSGAEVKKPGESLRISCKGS | GAAGTGCAGCTGGTGCAGTCTGGAGCAGAGGT |
| (type a) | (SEQ ID NO: 147) | GAAAAAGCCCGGGGAGTCTCTGAGGATCTCCT |
| | | GTAAGGGTTCT (SEQ ID NO: 148) |
| | | GAAGTGCAGCTGGTGCAGTCTGGCGCCGAAGT |
| | | GAAGAAGCCTGGCGAGTCCCTGCGGATCTCCT |
| | | GCAAGGGCTCT (SEQ ID NO: 149) |
| | | GAGGTGCAGCTGGTGCAGTCAGGCGCCGAAGT |
| | | GAAGAAGCCCGGCGAGTCACTGAGAATTAGCT |
| | | GTAAAGGTTCA (SEQ ID NO: 150) |
| VHFW1 | QVQLVQSGAEVKKPGASVKVSCKA | CAGGTTCAGCTGGTGCAGTCTGGAGCTGAGGT |
| (type b) | S (SEQ ID NO: 151) | GAAGAAGCCTGGGGCCTCAGTGAAGGTCTCCT |
| | | GCAAGGCTTCT (SEQ ID NO: 152) |
| VHFW2 | WVRQATGQGLEWMG | TGGGTGCGACAGGCCACTGGACAAGGGCTTGA |
| (type a) | (SEQ ID NO: 153) | GTGGATGGGT (SEQ ID NO: 154) |
| | | TGGGTGCGACAGGCTACCGGCCAGGGCCTGGA |
| | | ATGGATGGGC (SEQ ID NO: 155) |
| | | TGGGTCCGCCAGGCTACCGGTCAAGGCCTCGA |
| | | GTGGATGGGT (SEQ ID NO: 156) |
| VHFW2 | WIRQSPSRGLEWLG | TGGATCAGGCAGTCCCCATCGAGAGGCCTTGA |
| (type b) | (SEQ ID NO: 157) | GTGGCTGGGT (SEQ ID NO: 158) |
| | | TGGATCCGGCAGTCCCCCTCTAGGGGCCTGGA |
| | | ATGGCTGGGC (SEQ ID NO: 159) |
| VHFW2 | WVRQAPGQGLEWMG | TGGGTGCGACAGGCCCCTGGACAAGGGCTTGA |
| (type c) | (SEQ ID NO: 160) | GTGGATGGGT (SEQ ID NO: 161) |
| VHFW3 | RVTITADKSTSTAYMELSSLRSEDTA | AGAGTCACGATTACCGCGGACAAATCCACGAGC |
| (type a) | VYYCTR (SEQ ID NO: 162) | ACAGCCTACATGGAGCTGAGCAGCCTGAGATCT |
| | | GAGGACACGGCCGTGTATTACTGTACAAGA |
| | | (SEQ ID NO: 163) |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGG |
| | | (SEQ ID NO: 164) |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGG |
| | | (SEQ ID NO: 165) |
| VHFW3 | RFTISRDNSKNTLYLQMNSLRAEDT | AGATTCACCATCTCCAGAGACAATTCCAAGAAC |
| (type b) | AVYYCTR (SEQ ID NO: 166) | ACGCTGTATCTTCAAATGAACAGCCTGAGAGCC |
| | | GAGGACACGGCCGTGTATTACTGTACAAGA |
| | | (SEQ ID NO: 167) |
| | | AGGTTCACCATCTCCCGGGACAACTCCAAGAAC |
| | | ACCCTGTACCTGCAGATGAACTCCCTGCGGGCC |
| | | GAGGACACCGCCGTGTACTACTGTACCAGA |
| | | (SEQ ID NO: 168) |
| VHFW4 | WGQGTTVTVSS | TGGGGCCAGGGCACCACCGTGACCGTGTCCTC |
| | (SEQ ID NO: 169) | C (SEQ ID NO: 170) |
| | | TGGGGCCAGGGCACCACAGTGACCGTGTCCTC |
| | | T (SEQ ID NO: 171) |
| | | TGGGGTCAAGGCACTACCGTGACCGTGTCTAG |
| | | C (SEQ ID NO: 172) |
| | | TGGGGCCAGGGCACAACAGTGACCGTGTCCTC |
| | | C (SEQ ID NO: 173) |
| VLFW1 | EIVLTQSPDFQSVTPKEKVTITC | GAAATTGTGCTGACTCAGTCTCCAGACTTTCAGT |
| (type a) | (SEQ ID NO: 174) | CTGTGACTCCAAAGGAGAAAGTCACCATCACCT |
| | | GC (SEQ ID NO: 175) |
| | | GAGATCGTGCTGACCCAGTCCCCCGACTTCCAG |
| | | TCCGTGACCCCCAAAGAAAAAGTGACCATCACA |
| | | TGC (SEQ ID NO: 176) |
| VLFW1 | EIVLTQSPATLSLSPGERATLSC | GAAATTGTGTTGACACAGTCTCCAGCCACCCTG |
| (type b) | (SEQ ID NO: 177) | TCTTTGTCTCCAGGGGAAAGAGCCACCCTCTCC |
| | | TGC (SEQ ID NO: 178) |
| | | GAGATCGTGCTGACCCAGTCCCCTGCCACCCT |
| | | GTCACTGTCTCCAGGCGAGAGAGCTACCCTGTC |
| | | CTGC (SEQ ID NO: 179) |
| | | GAGATCGTCCTGACTCAGTCACCCGCTACCCTG |
| | | AGCCTGAGCCCTGGCGAGCGGGCTACACTGAG |
| | | CTGT (SEQ ID NO: 180) |
| VLFW1 | DIVMTQTPLSLPVTPGEPASISC | GATATTGTGATGACCCAGACTCCACTCTCCCTG |
| (type c) | (SEQ ID NO: 181) | CCCGTCACCCCTGGAGAGCCGGCCTCCATCTC |
| | | CTGC (SEQ ID NO: 182) |
| VLFW1 | DVVMTQSPLSLPVTLGQPASISC | GATGTTGTGATGACTCAGTCTCCACTCTCCCTG |
| (type d) | (SEQ ID NO: 183) | CCCGTCACCCTTGGACAGCCGGCCTCCATCTCC |
| | | TGC (SEQ ID NO: 184) |
| VLFW1 | DIQMTQSPSSLSASVGDRVTITC | GACATCCAGATGACCCAGTCTCCATCCTCCCTG |
| (type e) | (SEQ ID NO: 185) | TCTGCATCTGTAGGAGACAGAGTCACCATCACT |
| | | TGC (SEQ ID NO: 186) |
| VLFW2 | WYQQKPGQAPRLLIY | TGGTACCAGCAGAAACCTGGCCAGGCTCCCAG |
| (type a) | (SEQ ID NO: 187) | GCTCCTCATCTAT (SEQ ID NO: 188) |
| | | TGGTATCAGCAGAAGCCCGGCCAGGCCCCCAG |
| | | ACTGCTGATCTAC (SEQ ID NO: 189) |
| | | TGGTATCAGCAGAAGCCCGGTCAAGCCCCTAGA |
| | | CTGCTGATCTAC (SEQ ID NO: 190) |
| VLFW2 | WYQQKPGKAPKLLIY | TGGTATCAGCAGAAACCAGGGAAAGCTCCTAAG |
| (type b) | (SEQ ID NO: 191) | CTCCTGATCTAT (SEQ ID NO: 192) |
| | | TGGTATCAGCAGAAGCCCGGTAAAGCCCCTAAG |
| | | CTGCTGATCTAC (SEQ ID NO: 193) |
| VLFW2 | WYLQKPGQSPQLLIY | TGGTACCTGCAGAAGCCAGGGCAGTCTCCACA |
| (type c) | (SEQ ID NO: 194) | GCTCCTGATCTAT (SEQ ID NO: 195) |
| VLFW3 | GVPSRFSGSGSGTDFTFTISSLEAE | GGGGTCCCCTCGAGGTTCAGTGGCAGTGGATC |
| (type a) | DAATYYC (SEQ ID NO: 196) | TGGGACAGATTTCACCTTTACCATCAGTAGCCT |
| | | GGAAGCTGAAGATGCTGCAACATATTACTGT |
| | | (SEQ ID NO: 197) |
| | | GGCGTGCCCTCTAGATTCTCCGGCTCCGGCTCT |
| | | GGCACCGACTTTACCTTCACCATCTCCAGCCTG |
| | | GAAGCCGAGGACGCCGCCACCTACTACTGC |
| | | (SEQ ID NO: 198) |
| | | GGCGTGCCCTCTAGGTTTAGCGGTAGCGGTAG |
| | | TGGCACCGACTTCACCTTCACTATCTCTAGCCT |
| | | GGAAGCCGAGGACGCCGCTACCTACTACTGT |
| | | (SEQ ID NO: 199) |
| VLFW3 | GIPPRFSGSGYGTDFTLTINNIESED | GGGATCCCACCTCGATTCAGTGGCAGCGGGTA |
| (type b) | AAYYFC (SEQ ID NO: 200) | TGGAACAGATTTTACCCTCACAATTAATAACATA |
| | | GAATCTGAGGATGCTGCATATTACTTCTGT (SEQ |
| | | ID NO: 201) |
| VLFW3 | GVPSRFSGSGSGTEFTLTISSLQPD | GGGGTCCCATCAAGGTTCAGCGGCAGTGGATC |
| (type c) | DFATYYC (SEQ ID NO: 202) | TGGGACAGAATTCACTCTCACCATCAGCAGCCT |
| | | GCAGCCTGATGATTTTGCAACTTATTACTGT |
| | | (SEQ ID NO: 203) |
| | | GGCGTGCCCTCTAGATTCTCCGGCTCCGGCTCT |
| | | GGCACCGAGTTTACCCTGACCATCTCCAGCCTG |
| | | CAGCCCGACGACTTCGCCACCTACTACTGC |
| | | (SEQ ID NO: 204) |
| VLFW3 | GVPSRFSGSGSGTDFTFTISSLQPE | GGGGTCCCATCAAGGTTCAGTGGAAGTGGATCT |
| (type d) | DIATYYC (SEQ ID NO: 205) | GGGACAGATTTTACTTTCACCATCAGCAGCCTG |
| | | CAGCCTGAAGATATTGCAACATATTACTGT (SEQ |
| | | ID NO: 206) |
| | | GGCGTGCCCTCTAGGTTTAGCGGTAGCGGTAG |
| | | TGGCACCGACTTCACCTTCACTATCTCTAGCCT |
| | | GCAGCCCGAGGATATCGCTACCTACTACTGT |
| | | (SEQ ID NO: 207) |
| VLFW4 | FGQGTKVEIK (SEQ ID NO: 208) | TTCGGCCAAGGGACCAAGGTGGAAATCAAA |
| | | (SEQ ID NO: 209) |
| | | TTCGGCCAGGGCACCAAGGTGGAAATCAAG |
| | | (SEQ ID NO: 210) |
| | | TTCGGTCAAGGCACTAAGGTCGAGATTAAG |
| | | (SEQ ID NO: 211) |

TABLE 3
Constant region amino acid sequences of human IgG heavy chains and human
kappa light chain

| | |
|---|---|
| HC | IgG4 (S228P) mutant constant region amino acid sequence (EU Numbering) |
| | ASTKGPSVFP LAPCSRSTSE STAALGCLVK DYFPEPVTVS WNSGALTSGV HTFPAVLQSS |
| | GLYSLSSVVT VPSSSLGTKT YTCNVDHKPS NTKVDKRVES KYGPPCPPCP APEFLGGPSV |
| | FLFPPKPKDT LMISRTPEVT CVVVDVSQED PEVQFNWYVD GVEVHNAKTK PREEQFNSTY |
| | RVVSVLTVLH QDWLNGKEYK CKVSNKGLPS SIEKTISKAK GQPREPQVYT LPPSQEEMTK |
| | NQVSLTCLVK GFYPSDIAVE WESNGQPENN YKTTPPVLDS DGSFFLYSRL |
| | TVDKSRWQEG NVFSCSVMHE ALHNHYTQKS LSLSLGK (SEQ ID NO: 212) |
| LC | Human kappa constant region amino acid sequence |
| | RTVAAPSVFI FPPSDEQLKS GTASVVCLLN NFYPREAKVQ WKVDNALQSG NSQESVTEQD |
| | SKDSTYSLSS TLTLSKADYE KHKVYACEVT HQGLSSPVTK |
| | SFNRGEC (SEQ ID NO: 213) |
| HC | IgG4 (S228P) mutant constant region amino acid sequence lacing C-terminal |
| | lysine (K) (EU Numbering) |
| | ASTKGPSVFP LAPCSRSTSE STAALGCLVK DYFPEPVTVS WNSGALTSGV HTFPAVLQSS |
| | GLYSLSSVVT VPSSSLGTKT YTCNVDHKPS NTKVDKRVES KYGPPCPPCP APEFLGGPSV |
| | FLFPPKPKDT LMISRTPEVT CVVVDVSQED PEVQFNWYVD GVEVHNAKTK PREEQFNSTY |
| | RVVSVLTVLH QDWLNGKEYK CKVSNKGLPS SIEKTISKAK GQPREPQVYT LPPSQEEMTK |
| | NQVSLTCLVK GFYPSDIAVE WESNGQPENN YKTTPPVLDS DGSFFLYSRL |
| | TVDKSRWQEG NVFSCSVMHE ALHNHYTQKS LSLSLG (SEQ ID NO: 214) |
| HC | IgG1 wild type |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVDVS HEDPEVKFNW YVDGVEVHNA KTKPREEQYN |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 215) |
| HC | IgG1 (N297A) mutant constant region amino acid sequence (EU Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVDVS HEDPEVKFNW YVDGVEVHNA KTKPREEQYA |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 216) |
| HC | IgG1 (D265A, P329A) mutant constant region amino acid sequence (EU |
| | Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPELLGG |
| | PSVFLFPPKP KDTLMISRTP EVTCVVVAVS HEDPEVKFNW YVDGVEVHNA KTKPREEQYN |
| | STYRVVSVLT VLHQDWLNGK EYKCKVSNKA LAAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 217) |
| HC | IgG1 (L234A, L235A) mutant constant region amino acid sequence (EU |
| | Numbering) |
| | ASTKGPSVFP LAPSSKSTSG GTAALGCLVK DYFPEPVTVS WNSGALTSGV HTFPAVLQSS |
| | GLYSLSSVVT VPSSSLGTQT YICNVNHKPS NTKVDKRVEP KSCDKTHTCP PCPAPEAAGG |
| | PSVFLFPPKP KDTLMISRTP EVTCVVVDVS HEDPEVKFNW YVDGVEVHNA KTKPREEQYN |
| | STYRVVSVLT VLHQDWLNGK EYKCKVSNKA LPAPIEKTIS KAKGQPREPQ VYTLPPSREE |
| | MTKNQVSLTC LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 218) |

TABLE 4
Amino acid sequences of the heavy and light
chain leader sequences for humanized mAbs BAP049-
Clone-B and BAP049-Clone-E

| | | |
|---|---|---|
| BAP049- | HC | MAWVWTLPFLMAAAQSVQA (SEQ ID NO: 221) |
| Clone-B | LC | MSVLTQVLALLLLWLTGTRC (SEQ ID NO: 222) |
| BAP049- | HC | MAWVWTLPFLMAAAQSVQA (SEQ ID NO: 221) |
| Clone-E | LC | MSVLTQVLALLLLWLTGTRC (SEQ ID NO: 222) |

### EXAMPLES

### Examples 1-7: Examples of Low-Fat Meal

The examples below are for guidance only; meals are not restricted to the examples provided. Low-fat meal means approximately 100-500 calories and 1.5-15 grams of fat.

| | amount | kcal | total fat (g) |
|---|---|---|---|
| Example 1 | | | |
| wheat bread or toast | 2 slices | 134 | 1.7 |
| low-fat margarine | 1 tablespoon | 59 | 6.6 |
| jam, preserves, all flavors | 1 tablespoon | 55 | trace |
| skim milk | 1 cup | 86 | 0.4 |
| | Total: | 334 | 8.7 |
| Example 2 | | | |
| wheat bread or toast | 2 slices | 134 | 1.7 |
| jam, preserves, all flavors | 1 tablespoon | 55 | trace |
| skim milk | 1 cup | 86 | 0.4 |
| | Total: | 275 | 2.1 |
| Example 3 | | | |
| Jell-OTM individual pudding cup | 3.63 oz. | 117.5 | 1.5 |
| (chocolate, chocolate/vanilla swirl or | | | |
| vanilla) | | | |
| | Total: | 117.5 | 1.5 |
| Example 4 | | | |
| fried egg | 1 large egg | 90 | 6.83 |
| salsa, red, cooked | 6 tablespoons | 26 | 0 |
| orange juice | 1 cup | 114 | 0 |
| skim milk | 1 cup | 86 | 0.4 |
| | Total: | 316 | 7.23 |
| Example 5 | | | |
| ham, sliced, regular (~11% fat) | 2 slices (56 g) | 92 | 4.82 |
| cheese, Swiss | 1 oz. | 108 | 7.88 |
| toast, white | 1 slice | 64 | 0.88 |
| coffee, espresso, brewed | 1 oz. | 3 | 0.05 |
| | Total: | 267 | 13.63 |
| Example 6 | | | |
| toaster strudel | 1 strudel (53 g) | 197 | 9.8 |
| 2% milk | 1 cup | 125 | 4.7 |
| | Total: | 332 | 14.5 |
| Example 7 | | | |
| rice | 1 cup | 199 | 0.36 |
| fish (mackerel) | 1 oz. | 74 | 5.46 |
| pickles | 15 g (small) | 18 | 0.04 |
| skim milk | 1 cup | 86 | 0.4 |
| | Total: | 291 | 5.86 |

### (Reference) Example 8: Concomitant Administration of Ceritinib and Nivolumab from Cycle 1, Day 1

Combination therapy encompassed from the beginning of the combination treatment period Cycle 1 Day 1 concomitant administration of nivolumab 3 mg/kg Q2W+ceritinib, at assigned dose levels. Based on preliminary data, of the 36 patients enrolled to cohorts 1-4, the 450 mg dose level had higher rates of some adverse events, especially grade-3 rash, than expected based on data from either single agent. The proportion of patients with grade-3 rash was higher at the 450 mg dose level, 4/14 patients (29%), than at the 300 mg dose level, 3/22 patients (14%). Of the 14 patients treated at the 450 mg dose level, 9 (64%) achieved a confirmed partial response. Of the 22 patients treated at the 300 mg dose level, 4 (18%) achieved a confirmed complete (1 patient) or partial (3 patients) response; however, limited follow up, especially for cohort 4, confounds the assessment of confirmed response. The disease control rate (DCR) was similar for the 450 mg and 300 mg dose levels, 79% and 82% respectively. Thus, preliminary evidence of efficacy has been demonstrated at both tested dose levels. However, the 300 mg dose level appeared to have a lower number of objective responses, although this observation is based on a small number of patients with limited follow up. In addition, preliminary ceritinib steady state PK (AUC0-24 and Cmax) suggested that at 300 mg administered with food, exposure was approximately 60-65% that of 450 mg administered with food.

The observed incidence of rash was significantly more prevalent and severe than observed during the use of either single agent and thus needed to be addressed. Surprisingly, the incidence and grade of rash observed was higher in Asian population (93%) compared to Caucasian population (36%) (see tables 5 and 6). Hence, adapted dosing regimen that would reduce the rash problem would particularly suit the Asian population, although it would prove beneficial to the whole patient population (including Caucasians).

TABLE 5

| | Cohorts 1-4 | Cohorts 1-2 (450 mg ceritinib) | Cohort 3-4 (300 mg ceritinib) |
|---|---|---|---|
| Rash Max | N = 36 | N = 14 | N = 22 |
| Grade | n (%) | n (%) | n (%) |
| All grades | 21 (58%) | 8 (57%) | 13 (59%) |
| G3 | 7 (19%) | 4 (29%) | 3 (14%) |
| G2 | 7 (19%) | 3 (21%) | 4 (18%) |
| G1 | 7 (19%) | 1 (7%) | 6 (27%) |

TABLE 6

| | Cohorts 1-4 | Asian | Caucasian |
|---|---|---|---|
| | N = 36 | N = 14 | N = 22 |
| Rash Max Grade | n (%) | n (%) | n (%) |
| All grades | 21 (58%) | 13 (93%) | 8 (36%) |
| G3 | 7 (19%) | 3 (21%) | 4 (18%) |
| G2 | 7 (19%) | 6 (43%) | 1 (5%) |
| G1 | 7 (19%) | 4 (29%) | 3 (14%) |

### Example 9: Ceritinib Monotherapy Run-in Period, Followed by Concomitant Administration of Nivolumab and Ceritinib

Approximately 20 patients are enrolled at a starting dose of 450 mg ceritinib monotherapy administered for 2 cycles in the run-in period, followed by combination therapy of nivolumab 3 mg/kg Q2W+ceritinib. The cycle Patients initiate the combination therapy only after meeting safety criteria as specified below. In the combination period, patients receive the ceritinib dose prescribed just prior to start of combination treatment with ceritinib+nivolumab. That is, ceritinib is administered at a dose of 450 mg or lower, if dose reduction due to toxicity is required during the run-in period. Up to 2 dose reductions of ceritinib are permitted during the monotherapy run-in period and combination treatment period. The ceritinib dose level for combination therapy is determined for each patient based on tolerability during the monotherapy run-in period; ceritinib is administered at a dose of 450 mg, if dose reduction was not required during the run-in period, or lower (300 mg or 150 mg), if dose reduction due to toxicity was required during the run-in period. In alternative, and preferably, the 375 mg dose level can be used as a first fed dosing dose reduction step from 450 mg. Up to two dose reductions of ceritinib are permitted during the run-in period. The intermittent dose reduction to 375 mg allows for retaining better efficacy while it can reduce side effects, including rash.

On days of combination therapy, ceritinib can be administered prior to nivolumab and its premedication (if any pre-medication is necessary). The sequence allows consistent time of daily dosing for ceritinib. A minimum of 1 hour is best to pass from the time of ceritinib administration to the administration of nivolumab.

After 2 cycles of ceritinib monotherapy treatment, patients are assessed for safety. Prior to initiating nivolumab therapy in the combination treatment period, patients must meet the following criteria:
Acceptable tolerability of ceritinib (e.g. all treatment related adverse events ≤Grade 2 and Aspartate aminotransaminase (AST)≤3×ULN, Alanine aminotransaminase (ALT)≤3×ULN and Alkaline phosphatase (ALP)≤5.0×ULN, rash of any grade, which requires delay of combination therapy until resolution to Grade 0. Pneumonitis of any grade leads to discontinuation of treatment.

Ceritinib monotherapy must be dosed for at least 7 consecutive days prior to the first dose of nivolumab. If ceritinib dose modification is required during the monotherapy run-in period, the patient must receive at least 7 days of ceritinib dosing at the reduced dose, and meet all safety criteria specified above, prior to the first administration of nivolumab. Patients who do not meet these criteria must delay initiation of combination therapy by up to 1 additional cycle (Run-in Cycle 3) until these safety criteria are met, but may continue to receive monotherapy ceritinib unless precluded by toxicity. Patients who do not meet the safety criteria to initiate combination therapy by run-in Cycle 3 Day 28 are not be eligible to proceed to the combination therapy period, but may continue to receive monotherapy ceritinib in the monotherapy run-in period until the patient experiences unacceptable toxicity that precludes further treatment, disease progression (patients may continue to receive treatment with ceritinib following disease progression if, in the opinion of the physician, continued treatment would provide clinical benefit).

Evaluable patients for the determination of the MTD (Maximum Tolerated Dose) (dose-determining set, DDS) are defined as patients who have met the minimum safety evaluation requirements of the study as follows:

Patients who have received at least one dose of ceritinib and nivolumab in the dose escalation phase of the study and who

1) either meet the minimum exposure criteria and have sufficient safety evaluations, or
2) have experienced a dose limiting toxicity during the first 6 weeks after starting combination treatment with ceritinib and nivolumab. Minimum exposure criteria are a minimum of 28 days of treatment with ceritinib and 2 complete nivolumab infusions during the first 6 weeks after starting combination therapy.

Patients who do not meet these minimum safety evaluation requirements will be regarded as ineligible for the DDS and additional patients may be enrolled to the cohort. For patients who meet these criteria, the final assessment of evaluability for determination of the MTD are determined based on the available PK data and other relevant data. Additional patients may be enrolled in order to ensure that the minimum number of evaluable patients per cohort for the primary objective is met.

Primary objective is to determine the maximum tolerated dose (MTD) of combination ceritinib and nivolumab and to assess the preliminary anti-tumor activity of the ceritinib and nivolumab combination at the recommended dose for expansion.

The design of the study can be an open-label, multi-center dose escalation and expansion study investigating the safety and tolerability, PK/PD, and preliminary efficacy of combination ceritinib and nivolumab for the treatment of patients with metastatic, ALK-positive cancer, such as non-small cell lung cancer (NSCLC). Patients enrolled are histologically or cytologically confirmed diagnosis of a cancer that carries an ALK rearrangement as determined by the FDA approved test. The cancer can be NSCLC.

Treatment with ceritinib and nivolumab may continue until the patient experiences unacceptable toxicity that precludes further treatment, disease progression (patients may continue to receive treatment with ceritinib and/or nivolumab following disease progression if, in the opinion of the physician, continued treatment would provide clinical benefit), withdrawal of consent, and/or at the discretion of the physician. If, due to toxicity, a patient requires discontinuation of only one of the study drugs, the patient may continue in the study while continuing to receive only the tolerated study drug. This additional treatment will provides data to support the secondary and exploratory study objectives of assessing safety, tolerability, and antitumor activity of combination ceritinib and nivolumab.

The toxicity is deemed to be at least initiated by ceritinib. Therefore, once confined and properly handled by reducing the dose of ceritinib, nivolumab can be added. Since the mechanism of action is expected to be the same for nivolumab as for other anti-PD-1 antibody molecules, and anti-PD-1 antibody molecules bind to specific targets, i.e. are very selective, it can be easily contemplated that the same beneficial effect of the new dosing regimen, which includes a 2-3 run-in cycles of ceritinib monotherapy, would be observed with other combinations of ceritinib and anti-PD-1 antibody molecule.

1. A pharmaceutical combination comprising (i) ceritinib or a pharmaceutically acceptable salt thereof, and (ii) anti-PD-1 antibody molecule or a pharmaceutically acceptable salt thereof, for use in the treatment of cancer, wherein (i) ceritinib is administered as monotherapy for 2 cycles before a combination therapy with the (ii) anti-PD-1 antibody molecule is initiated.

2. The pharmaceutical combination of claim 1, wherein the (ii) anti-PD-1 antibody molecule is initiated only in a patient without rash of any grade.

3. The pharmaceutical combination of claim 1, wherein the (ii) anti-PD-1 antibody molecule is initiated only in a patient that has Aspartate aminotransaminase (AST)≤3×ULN, Alanine aminotransaminase (ALT)≤3×ULN and Alkaline phosphatase (ALP)≤5.0×ULN.

4. The pharmaceutical combination of claim 1, wherein (i) ceritinib is administered as monotherapy for 3 cycles before a combination therapy with the (ii) anti-PD-1 antibody molecule is initiated.

5. The pharmaceutical combination of claim 1, wherein (i) ceritinib is administered for at least 7 consecutive days prior to the first dose of the (ii) anti-PD-1 antibody molecule.

6. The pharmaceutical combination of claim 1, wherein (i) ceritinib is administered at a starting dose of 450 mg ceritinib daily together with a low-fat meal.

7. The pharmaceutical combination of claim 1, wherein the ceritinib starting dose is reduced to 375 mg, 300 mg or 150 mg daily.

8. The pharmaceutical combination of claim 1, wherein the ceritinib starting dose is reduced to 375 mg.

9. The pharmaceutical combination of claim 1, wherein the cancer is ALK-positive cancer.

10. The pharmaceutical combination of claim 9, wherein the ALK-positive cancer is NSCLC.

11. The pharmaceutical combination of claim 1, wherein the (ii) anti-PD-1 antibody molecule is selected from nivolumab, pembrolizumab, pidilizumab, PDR-001, or a pharmaceutical salt thereof.

12. The pharmaceutical combination of claim 11, wherein the (ii) anti-PD-1 antibody molecule is administered every 2 weeks, every 3 weeks or every 4 weeks.

13. The pharmaceutical combination of claim 11, wherein the (ii) anti-PD-1 antibody molecule is nivolumab and 3 mg/kg nivolumab is administered every 2 weeks.

14. The pharmaceutical combination of claim 11, wherein the (ii) anti-PD-1 antibody molecule is PDR-001, or a pharmaceutical salt thereof.

15. The pharmaceutical combination of claim 14, wherein PDR-001 is administered intravenously in a single dose of 300 to 400 mg/day.

16. The pharmaceutical combination of claim 1, wherein the patient is Asian.

17. A method for the treatment of cancer in a patient in need thereof, comprising administering to the patient a therapeutically effective amount of the pharmaceutical combination of claim 1.

18. (canceled)

19. The method of claim 17, wherein the cancer is ALK-positive cancer.

20. The method of claim 19, wherein the ALK-positive cancer is NSCLC.
